(19) United States
(12) Patent Application Publication
(10) Pub. No.: US 2023/0053469 A1
(43) Pub. Date: Feb. 23, 2023

(54) Automated Suggestions and Monitoring in a Longitudinal Care Plan

(71) Applicant: CERNER INNOVATION, INC., KANSAS CITY, KS (US)

(72) Inventors: Sara Boswell, Blue Springs, MO (US); Kelsey O'Keefe, Kansas City, MO (US); Manoj Mylapore Chandrasekaran, Overland Park, KS (US); John Scheeser, Blue Springs, MO (US)

(21) Appl. No.: 17/404,138

(22) Filed: Aug. 17, 2021

(51) Int. Cl. G16H 20/00; G16H 15/00; G16H 80/00

(52) U.S. Cl. CPC G16H 20/00; G16H 15/00; G16H 80/00

(57) ABSTRACT

Systems, methods, and user interfaces are provided for providing automated suggestions to longitudinal care plans of patients based on gaps in care and automatically updating measures, goals, and/or activities that represent the gaps in care based on changes to registries or measure outcomes corresponding to the patients. After accessing a registry corresponding to a particular condition of a patient, it can be determined that the patient has a gap in care corresponding to the particular condition in a longitudinal care plan of a patient. At least one measure that addresses the gap in care can be identified and created on the longitudinal care plan of the patient. By monitoring a status of the measure for the patient, it can be determined the patient has completed the at least measure that addresses the gap in care. Based on the completed at least one measure, the longitudinal care plan of the patient can be updated.

### BACKGROUND

For all patients, there is a recommended set of care activities that should be delivered based on background, demographics, and overall health. Examples might include getting an annual flu shot for most adults, getting an annual HbA1c measurement for a diabetic, or getting a colonoscopy every five years for people over fifty. Any deviation from these recommended set of care activities is considered a “gap in care” or a care gap. Closing gaps in care is important, especially for health systems aligning to value-based payment models, since it ensures a consistency of care that is based on current clinical best practices for the patient, potentially improving outcomes for the patient population, and decreasing overall cost. Ensuring that gaps in care are closed should be a priority for all patients and their respective care teams.

Previous solutions to this problem focused on specific roles and workflows (such as providers or care managers). As a result, certain members of the care team, including the patient, are often overlooked. Because not everyone on the care team, or the patient, is exposed to care activities, care gaps remain an obstacle to consistency of care, improved outcomes, and rising costs in health care.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present disclosure relate to systems, methods, and user interfaces for providing automated suggestions and monitoring in a longitudinal care plan. More particularly, embodiments of the present disclosure provides automated suggestions to longitudinal care plans of patients based on gaps in care and automatically updating measures that represent the gaps in care based on changes to registries or measure outcomes corresponding to the patients. After accessing a registry corresponding to a particular condition of a patient, it can be determined that the patient has a gap in care corresponding to the particular condition in a longitudinal care plan of a patient. At least one measure that addresses the gap in care can be identified and created on the longitudinal care plan of the patient. By monitoring the gap in care for the patient, it can be determined the patient has completed the at least measure that addresses the gap in care. Based on the measure state, the longitudinal care plan of the patient can be updated.

### BRIEF DESCRIPTION OF THE SEVERAL VIEWS OF THE DRAWINGS

The patent or application file contains at least one drawing executed in color. The present invention is described in detail below with reference to the attached drawing figures, wherein:

FIG. 1 is a block diagram of an exemplary operating environment suitable to implement embodiments of the present invention;

FIG. 2 depicts an exemplary framework of a longitudinal care plan system suitable to implement embodiments of the present invention;

FIG. 3 depicts the flow of information between various components of a longitudinal care plan system suitable to implement embodiments of the present invention;

FIG. 4 depicts a flow diagram illustrating a method of adding a measure to a longitudinal care plan, in accordance with an embodiment of the present invention;

FIG. 5 depicts a flow diagram illustrating a method of monitoring a status of a measure in a longitudinal care plan, in accordance with an embodiment of the present invention;

FIG. 6 depicts a flow diagram illustrating a method of updating a status of a measure in a longitudinal care plan, in accordance with an embodiment of the present invention;

FIGS. 7-14 depict illustrative screen displays of longitudinal care plan interfaces, in accordance with an embodiment of the present invention;

FIG. 15 depicts a flow diagram illustrating a method of creating a measure in a longitudinal care plan, in accordance with an embodiment of the present invention; and

FIG. 16 depicts a flow diagram illustrating a method of updating a status of a measure corresponding to a measure in a longitudinal care plan, in accordance with an embodiment of the present invention.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” might be used herein to connote different elements of methods employed, the terms should not be interpreted as implying any particular order among or between various steps herein disclosed unless and except when the order of individual steps is explicitly stated.

Various terms are used throughout this description. Definitions of some terms are included below to provide a clearer understanding of the ideas disclosed herein:

A consumer, as used herein, is a person, or the single, population-agnostic representation of a person in the context of a client.

Longitudinal care plan refers to an adaptive, intelligent, cross-system, cross-venue, and cross-discipline plan to facilitate personalized health management and care delivery to an optimal outcome. A person's longitudinal care plan is not a concrete entity in and of itself and has no attributes. Rather, it is a collection of related entities such as health concerns, goals, activities, etc. As such, a person has a single longitudinal care plan.

A health concern is a health focus of a person that is of concern and actively being treated or managed. A health concern may correspond to a documented condition or risk (which may be documented or derived); however, not every condition or risk will be elevated to the level of a health concern to be managed. Only those conditions or risks that are longitudinally managed by the care team (i.e., have current or historical goals, activities, etc.) will be represented by a health concern. The system allows for adding health concerns that do not correspond to documented activity, as well as non-codified health concerns (i.e., “free-text” health concerns). Examples of health concerns include conditions (e.g., diabetes, heart failure, etc.), risks (e.g., fall risk), pregnancy, wellness, social determinants (e.g., access to food, lack of transportation, etc.), and the like.

A goal specifies a future target or achievement towards which the effort of care planning and execution is directed. Goals represent concrete targets to reduce or eliminate concerns or risks. A goal may exist in the absence of concerns or risks. For example, a patient may have a goal to improve his or her fitness level. Goals may or may not measurable. Measurable goals (also called targets) contain a target value to be met in order for the goal to be achieved. Non-measurable goals may still have a start and stop date, duration, etc. Additionally, goals may be short-term or long-term in nature. Goals can be used to support or make progress on addressing a defined health concern. Goals can also be associated to one or more health concerns. For example, a goal of “Stop Smoking” could help support or improve multiple health concerns. Goals can also be setup as short-term goals that support another goal. Examples of measurable goals may include: maintain HbA1c<7.5%, lose 10 pounds in four weeks, walk ten thousand steps per day, obtain relief from back pain, and the like. Examples of non-measure goals may include: increase strength and endurance, walk with a steady gait, remain free from drinking alcohol, and the like.

An activity refers to an action that is carried out to support or reinforce the patient's health and wellness. Activities can support and be associated with one or more health concerns. Activities can also support and be associated with one or more goals. Examples of activities may include: medication (e.g., take 500 mg Metformin two times daily), monitoring (e.g., use blood pressure device daily), assessment (e.g., take the Asthma Control Test (ACT)), education (e.g., enroll in diabetes education course), instruction (e.g., use a shower bench when bathing), physical (e.g., exercise three times a week), and the like.

A measure refers to an activity or goal that specifically addresses a gap in care identified by the longitudinal care plan system, as described herein.

A care team is the set of people who are actively managing and/or making decisions about a person's health. Some care team members may be represented as personnel or consumers, and can interact with the longitudinal care plan directly through solutions, whereas other members (such as power of attorney, some family members) may not. Examples of care team members include: primary care provider, care manager or care coordinator, specialist, person or consumer (i.e., the subject of the plan), power of attorney, legal proxy, family member, and the like.

As noted in the Background, for all patients, there is a recommended set of care activities that should be delivered based on background, demographics, and overall health. Examples might include getting an annual flu shot for most adults, getting an annual HbA1c measurement for a diabetic, or getting a colonoscopy every five years for people over 50. Any deviation from these recommended set of care activities is considered a “gap in care” or a care gap. Closing gaps in care is important, especially for health systems aligning to value-based payment models, since it ensures a consistency of care that is based on current clinical best practices for the patient, potentially improving outcomes for the patient population, and decreasing overall cost. Ensuring that gaps in care are closed should be a priority for all patients and their respective care teams.

Previous solutions to this problem focused on specific roles and workflows (such as providers or care managers). As a result, certain members of the care team, including the patient, are often overlooked. Because not everyone on the care team, or the patient, is exposed to care activities, care gaps remain an obstacle to consistency of care, improved outcomes, and rising costs in health care.

Embodiments of the present disclosure relate to systems, methods, and user interfaces for providing automated suggestions and monitoring in a longitudinal care plan. More particularly, embodiments of the present disclosure provides automated suggestions to longitudinal care plans of patients based on gaps in care and automatically updating statuses of measures that address the gaps in care based on changes to registries or measure outcomes corresponding to the patients. After accessing a registry corresponding to a particular condition of a patient, it can be determined that the patient has a gap in care corresponding to the particular condition in a longitudinal care plan of a patient. At least one measure that addresses the gap in care can be identified and created on the longitudinal care plan of the patient. By monitoring a status of the at least one measure for the patient, it can be determined the patient has completed the at least measure that addresses the gap in care. Based on the status of the measure, the longitudinal care plan of the patient can be updated.

These features enable clients to create and manage connections between longitudinal care plan goals and/or activities and various registries and measures. By establishing these connections, if a particular measure is placed on a patient's longitudinal care plan, changes to the patient's outcomes for the registry or measure connected to that measure may update various attributes on that measure. By way of example, assume a patient is on a general wellness registry, with an activity for “Annual Office Visit” that the patient is not currently meeting. The client may create an activity definition in their reference data called “Complete an annual physical” and associate that activity with the “Annual Office Visit” measure. If the “Complete an annual physical” activity were added to the patient's Longitudinal care plan, and later the patient were to meet the “Annual Office Visit” measure (the status on the measure changed from “Not Met” to “Met”), the status of the associated activity on the patient's plan would be updated to “Completed.”

Integrating these measures to the longitudinal care plan based on a patient's care gaps is beneficial for a number of reasons. First, the friction is removed from creating and managing a patient's care plan. Additionally, by including suggestions in the care plan, the care gaps are exposed in a platform that is used by all members of a patient's care team, instead of only being exposed to certain roles and workflows. Moreover, by creating goal and/or activity suggestions based on care gaps, the care gaps are translated into clear, actionable items (i.e., the measures) that all members of the patient's care team can act on as needed and consistency can be maintained across various roles and workflows. Finally, by including care gaps in the patient's longitudinal care plan, the care gaps are exposed to the patient in various patient-facing workflows, increasing the patient's awareness of the care gaps, and providing guidance on how to address the care gaps. In these ways, everyone on the care team, including the patient, is exposed to care activities, care gaps are no longer an obstacle to consistency of care, outcomes are improved, and rising costs in health care can be prevented.

Accordingly, one embodiment of the present disclosure is directed to one or more computer storage media having computer-executable instructions embodied thereon that, when executed by a computer, causes the computer to perform operations. The operations include accessing a registry corresponding to a particular condition of a patient. The operations also include determining the patient has a gap in care corresponding to the particular condition in a longitudinal care plan of a patient. The operations further include identifying at least one measure that addresses the gap in care. The operations also include creating the at least one measure on the longitudinal care plan of the patient.

In another embodiment, the present disclosure directed to a computerized method. The method comprises monitoring a gap in care for a patient. The method also comprises determining the patient has completed at least one measure addressing the gap in care corresponding to a particular condition in the longitudinal care plan of the patient. The method further comprises, based on completing the at least one measure, updating the longitudinal care plan of the patient.

In yet another embodiment, the present disclosure is directed to a system. The system comprises: a processor; and a computer storage medium storing computer-usable instructions that, when used by the processor, cause the processor to: access a registry corresponding to a particular condition of a patient; determine the patient has a gap in care corresponding to the particular condition in a longitudinal care plan of a patient; identify at least one measure that addresses the gap in care; create the at least one measure on the longitudinal care plan of the patient; monitor the gap in care for the patient; determine the patient has completed the at least one measure that addresses the gap in care; and based on the completed at least one measure, update the longitudinal care plan of the patient.

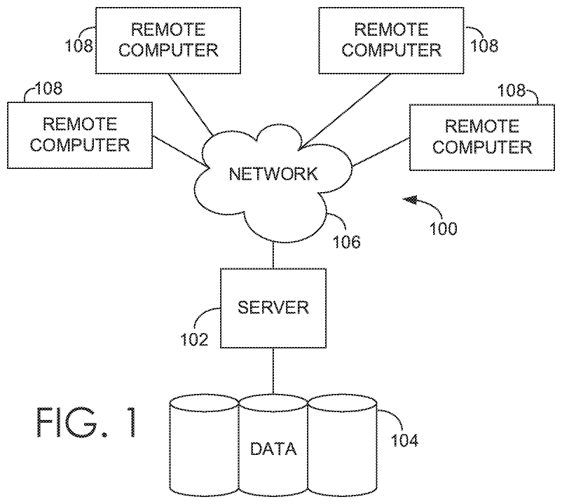

Having briefly described embodiments of the present invention, an exemplary operating environment suitable for use in implementing embodiments of the present invention is described below. FIG. 1 provides an aspect of an example operating environment with which embodiments of the present invention may be implemented. The aspect of an operating environment is illustrated and designated generally as reference numeral 100.

Example operating environment 100 comprises a general purpose computing device in the form of a control server 102. Exemplary components of the control server 102 comprise a processing unit, internal system memory, and a suitable system bus for coupling various system components, including database cluster 104, with the control server 102. The system bus might be any of several types of bus structures, including a memory bus or memory controller, a peripheral bus, and a local bus, using any of a variety of bus architectures. Exemplary architectures comprise Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

Control server 102 typically includes therein, or has access to, a variety of computer-readable media, for instance, database cluster 104. Computer-readable media can be any available media that might be accessed by control server 102, and includes volatile and nonvolatile media, as well as, removable and nonremovable media. Computer-readable media might include computer storage media. Computer storage media includes volatile and nonvolatile media, as well as removable and nonremovable media implemented in any method or technology for storage of information, such as computer-readable instructions, data structures, program modules, or other data. In this regard, computer storage media might comprise RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 102. Computer storage media does not comprise signals per se. Combinations of any of the above also may be included within the scope of computer-readable media.

The computer storage media discussed above and illustrated in FIG. 1, including database cluster 104, provide storage of computer-readable instructions, data structures, program modules, and other data for the control server 102. In some embodiments, data cluster 104 takes the form of a cloud-based data store, and in some embodiments is accessible by a cloud-based computing platform.

The control server 102 might operate in a computer network 106 using logical connections to one or more remote computers 108. Remote computers 108 might be located at a variety of locations in a medical or research environment, including clinical laboratories (e.g., molecular diagnostic laboratories), hospitals and other inpatient settings, veterinary environments, ambulatory settings, medical billing and financial offices, hospital administration settings, home health care environments, and providers' offices. Providers may comprise a treating physician or physicians; specialists such as surgeons, radiologists, cardiologists, and oncologists; emergency medical technicians; physicians' assistants; nurse practitioners; nurses; nurses' aides; pharmacists; dieticians; microbiologists; laboratory experts; laboratory technologists; genetic counselors; researchers; veterinarians; students; and the like.

The remote computers 108 might also be physically located in nontraditional medical care environments so that the entire health care community might be capable of integration on the network. The remote computers 108 might be personal computers, servers, routers, network PCs, peer devices, other common network nodes, or the like and might comprise some or all of the elements described above in relation to the control server 102. The devices can be personal digital assistants or other like devices.

Exemplary computer networks 106 comprise local area networks (LANs) and/or wide area networks (WANs). Such networking environments are commonplace in offices, enterprise-wide computer networks, intranets, and the Internet. When utilized in a WAN networking environment, the control server 102 might comprise a modem or other means for establishing communications over the WAN, such as the Internet. In a networked environment, program modules or portions thereof might be stored in association with the control server 102, the database cluster 104, or any of the remote computers 108. For example, various application programs may reside on the memory associated with any one or more of the remote computers 108. It will be appreciated by those of ordinary skill in the art that the network connections shown are exemplary and other means of establishing a communications link between the computers (e.g., control server 102 and remote computers 108) might be utilized.

In operation, an organization might enter commands and information into the control server 102 or convey the commands and information to the control server 102 via one or more of the remote computers 108 through input devices, such as a keyboard, a pointing device (commonly referred to as a mouse), a trackball, or a touch pad. Other input devices comprise microphones, satellite dishes, scanners, or the like. Commands and information might also be sent directly from a remote health care device to the control server 102. In addition to a monitor, the control server 102 and/or remote computers 108 might comprise other peripheral output devices, such as speakers and a printer.

In some embodiments, control server 102 is a computing system or platform made up of one or more computing devices. Embodiments of control server 102 may be a distributed computing system, a centralized computing system, a single computer such as a desktop or laptop computer or a networked computing system. Thus, in some embodiments, control server 102 comprises a multi-agent computer system with software agents.

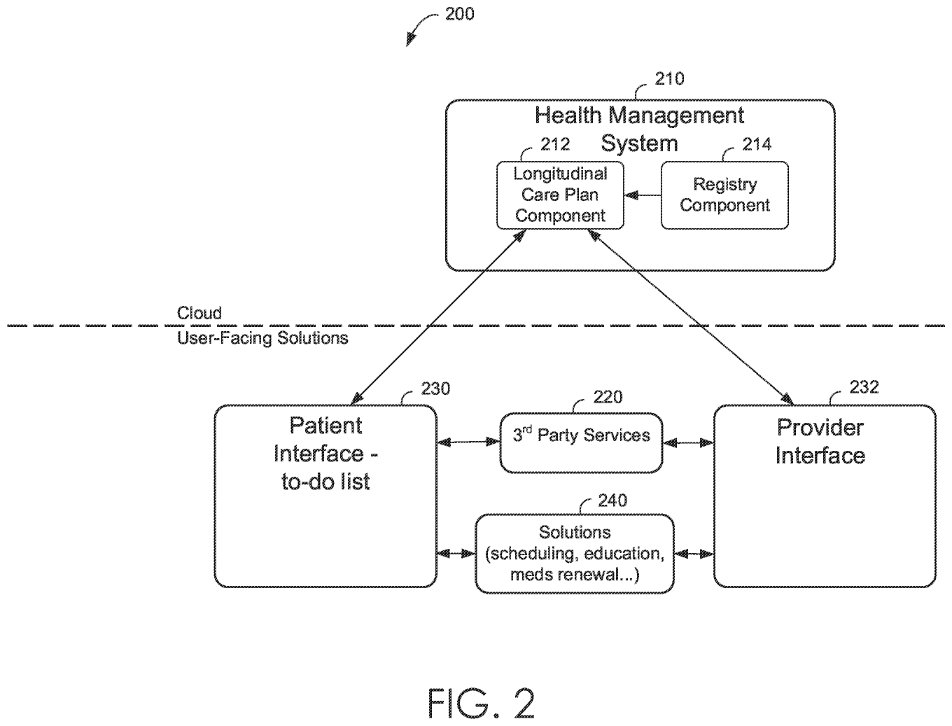

Turning now to FIG. 2, an exemplary framework of a longitudinal care plan system 200 is shown, in accordance with an aspect of the present invention. It should be understood that these and other arrangements described herein are set forth only as examples. Other arrangements and elements (e.g., machines, interfaces, functions, orders, and groupings of functions, etc.) can be used in addition to or instead of those shown, and some elements may be omitted altogether. Further, many of the elements described herein are functional entities that may be implemented as discrete or distributed components or in conjunction with other components, and in any suitable combination and location. Various functions described herein as being performed by one or more entities may be carried out by hardware, firmware, and/or software. For instance, various functions may be carried out by a processor executing instructions stored in memory. The longitudinal care plan system 200 may be implemented via any type of computing device, such as computing device 100 described above with reference to FIG. 1, for example.

Referring to the longitudinal care plan system 200 in FIG. 2, health management component 210 generally is a cloud-based, population health management system. Health management component 210 is vendor-agnostic and enables health care systems to aggregate, transform, and reconcile data sources across the continuum of care, creating a longitudinal health record for individual members of the population. Health management component comprises several components, a longitudinal care plan component 212 and a registry component 214.

Initially, registry component 214 provides longitudinal care plan component 212 with information corresponding to a particular aspect of an individual patient that is relevant to the care of that patient. For example, if a particular patient has been diagnosed with diabetes, registry component 214 may provide longitudinal care plan component 212 information corresponding to diabetes that includes standards of care to insure the patient is being managed appropriately for diabetes. The information may include educational information for the patient.

Longitudinal care plan component 212 proactively places various measures on a care plan (i.e., the longitudinal care plan) for the patient based on the information received from the registry component 214. Additionally, or alternatively, patient facing interface 230 communicates with longitudinal care plan component 212 APIs. For example, patient facing interface 230 may call a longitudinal care plan component 212 API to receive a to-do list from longitudinal care plan component 212. The to-do list may comprises various measures in the longitudinal care plan. The patient facing interface 230 may also include widgets that enable the patient to perform an action, such as completing a measure. For example, the patient facing interface 230 may enable the patient to schedule an appointment. The patient facing interface 230 may also display and/or update the status of a particular measure, activity, and/or goal, such as if the patient performs at least a portion of the measure. The patient facing interface 230 may also notify the patient when a measure is coming due. In embodiments, the patient facing interface 230 enables the patient to self-report the completion of a measure.

The patient facing interface 230 may communicate with a solutions component 240 that provides features such as scheduling, education, medications renewal, and the like, that are integrated into the patient facing interface 230 and assist the patient performing at least a portion of the activity or goal. The patient facing interface 230 may also communicate with third party services 220 including communication services, provider search services, scheduling services, social care services, survey and form services, and the like. Each of these third party services 220 may facilitate the patient completing measures that have been included on the care plan.

In some embodiments, provider facing interface 232 communicates with longitudinal care plan component 212 APIs. For example, provider facing interface 232 may call a longitudinal care plan component 212 API to receive the to-do list from longitudinal care plan component 212 so the provider is aware of the various measures in the longitudinal care plan. The provider facing interface 232 may also display and/or update the status of a particular measure, such as if the patient performs at least a portion of the measure. The provider facing interface 232 may also notify the provider when a measure is coming due. In embodiments, the provider facing interface 232 enables the provider to report the completion of a measure on behalf of the patient.

The provider facing interface 232 may communicate with a solutions component 240 that provides features such as scheduling, education, medications renewal, and the like, that are integrated into the provider facing interface 232 and assist the provider maintaining awareness of the patient's progress in performing at least a portion of the measure. The provider facing interface 232 may also communicate with third party services 220 including communication services, provider search services, scheduling services, social care services, survey and form services, and the like. Each of these third party services 220 may facilitate the provider maintaining awareness of the patient's progress in completing measures that have been included on the care plan.

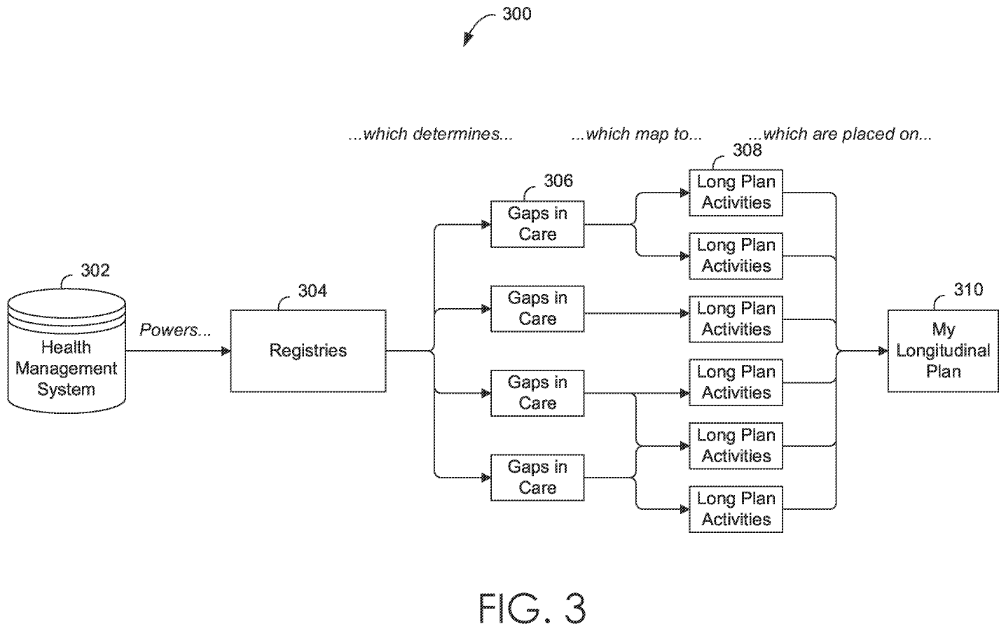

As shown in FIG. 3, depicts the flow of information between various components of the longitudinal care plan system 300. As illustrated, longitudinal care plan system 300 initially receives data at a health management system 302, such as Cerner Healthelntent®, from various sources (e.g., any electronic medical record, health information technology system, or other data sources including pharmacy benefits managers or insurance claims). Health management system 302 uses data from these sources to create longitudinal records for a plurality of patients in a population. Health management system 302 may also match individual patients or groups of patients in the populations to one or more registries 304. Based on information contained in the registries 304, gaps in care 306 may be automatically identified for a particular patient of the plurality of patients. As a result, long plan activities 308 (i.e., the measures) are automatically identified for the particular patient to address the gaps in care 306. Finally, long plan activities 308 are automatically added to the longitudinal care plan 310 for the patient. It should be noted that as the long plan activities 308 are addressed, the longitudinal care plan system 300 updates the longitudinal care plan 310.

The longitudinal care plan system facilitates creating the individual elements of the longitudinal care plan (goals, activities, strengths, etc.), creating relationships between the individual elements, and utilizing reference content that can be used to help users create meaningful codified plan elements. The relationship between plan elements and external resources can also be managed (e.g., health concerns).

In practice, the longitudinal care plan system is a cloud-based, cross-discipline, cross-venue care plan and is not confined to a single EMR or system. This is advantageous in that it allows users across different systems (such as different EMRs, patient portals, etc.) to view and interact with the plan, however, there are situations where some information may need to be saved in a local EMR and also represented in the longitudinal care plan. In these situations, manual double documentation can be reduced and information between both systems can be correlated with one another or created based on each other.

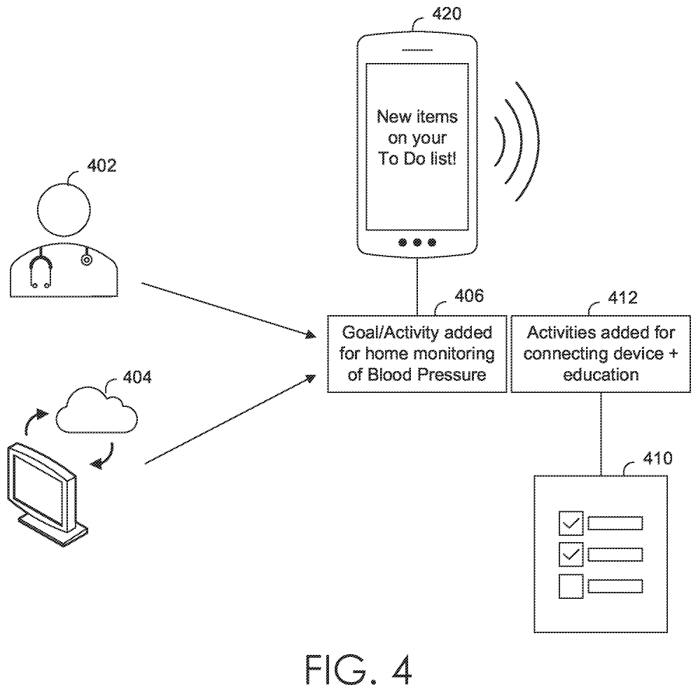

Turning now to FIG. 4, a flow diagram illustrates a method of adding a measure to a longitudinal care plan, in accordance with an embodiment of the present invention. As shown, a clinician 402 may add a measure 406 to the longitudinal care plan of a patient. Additionally or alternatively, a longitudinal care plan system (such as longitudinal care plan system 200 of FIG. 2 or longitudinal care plan system 300 of FIG. 3) may automatically add the measure 406 to the longitudinal care plan of the patient. By way of example, the measure 406 may be for the patient to monitor blood pressure at home. Accordingly, the measure is communicated in a to-do list to the patient device via a user interface 420.

Additionally or alternatively, measures 412 may be added to the longitudinal care plan of the patient for connecting a device corresponding to the measure for providing education to the patient. In this case, the measures 412 may correspond to connecting a blood pressure monitor and educating the patient on blood pressure (e.g., instructions on how to do so, the benefits of monitoring blood pressure, or actions the patient can take to improve blood pressure readings). Each of these measures may also be communicated in the to-do list to the patient device via the user interface 420. As the patient completes the measure, the longitudinal care plan system updates the to-do list 410.

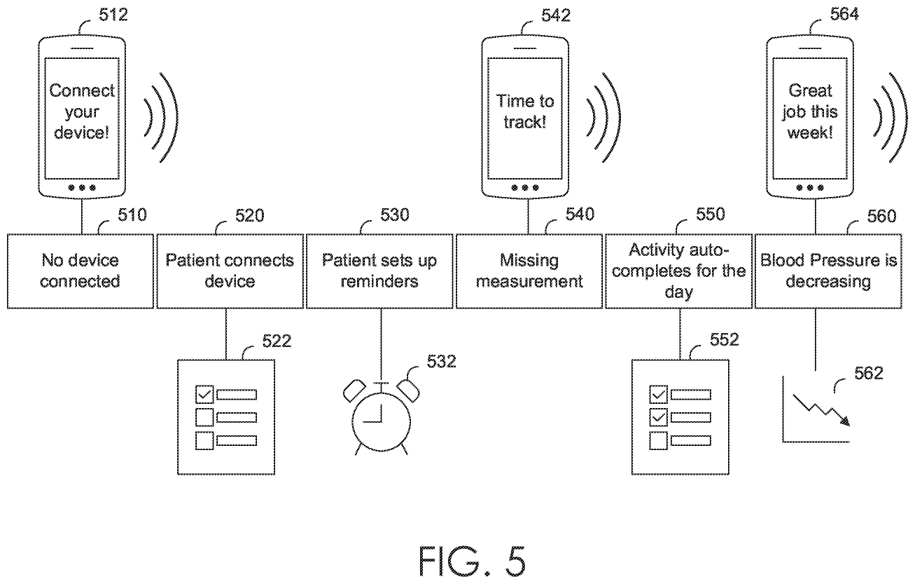

In FIG. 5, a flow diagram illustrates a method of monitoring a measure in a longitudinal care plan, in accordance with an embodiment of the present invention. As shown, FIG. 5 continues the example of FIG. 4. Initially, the longitudinal care plan system recognizes that the device 510 has not been connected. As the patient connects the device 520, the longitudinal care plan system updates the to-do list 522. The patient may set a reminder 530 to complete a measure, such as an alarm 532. If the patient has not recorded a measurement, the longitudinal care plan system identifies that there is a missing measurement 540, and an alert is provided via the user interface 542. When the measurement has been recorded, the longitudinal care plan system auto-completes the measure for the day 550 and updates the to-do list 552. Additionally or alternatively, the patient may self-report the measurement which also causes the longitudinal care plan system to update the to-do list 552. The longitudinal care plan system may recognize that the blood pressure is decreasing for the patient 560 based on a trend of recorded measurements 562. As a result, the longitudinal care plan system may provide recognition to the patient via the user interface 564.

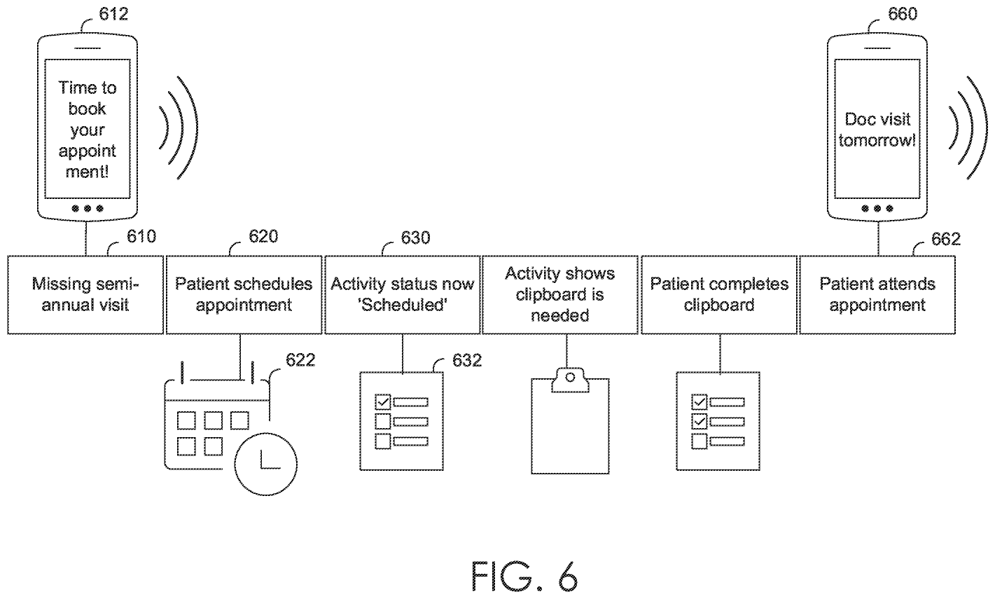

As illustrated in FIG. 6, a flow diagram illustrates a method of updating a measure in a longitudinal care plan, in accordance with an embodiment of the present invention. Initially, the longitudinal care plan system may recognize the patient has missed a semi-annual visit 610. Accordingly, the longitudinal care plan system provides an alert to the patient interface 612 indicating the gap in care. When the patient schedules the appointment 620, such as via a scheduling service 622 integrated with the longitudinal care plan system, the measure status is updated to “Scheduled” 630. The longitudinal care plan system updates the to-do list 632 to reflect the scheduled appointment. Accordingly, the longitudinal care plan system provides a reminder to the patient via the user interface 660 and the patient attends the appointment 662.

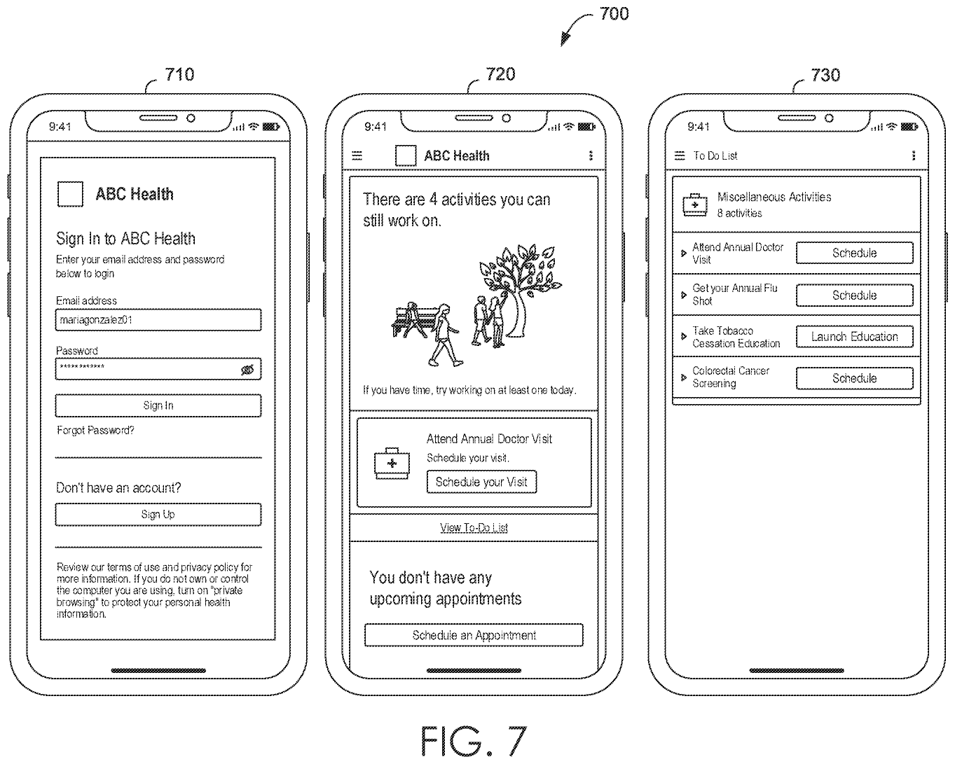

With reference to FIGS. 7-14, illustrative screen displays 700-1400 of embodiments of the present invention are shown. It is understood that each of the illustrative screen displays are connected logically, such that they comprise a user interface designed for a longitudinal care plan system, in accordance with embodiments of the present invention. The screen displays may appear in any order and with any number of screen displays, without regard to whether the screen display is described or depicted herein. The screen displays provide tools and features that provide automated suggestions and monitoring in a longitudinal care plan, in accordance with embodiments of the present invention.

In FIG. 7, screen displays 710-730 depict a progression of user interfaces of a longitudinal care plan system. For example, screen display 710 illustrates an initial login screen for the patient. Upon logging in, screen display 720 illustrates an overview of the longitudinal care plan. Initially, an activity display 722 may display a number of activities the patient can work on. A reminder display 724 may also indicate that the patient needs to schedule an annual doctor visit and may include a widget that enables the patient to do so. A to-do list button 726 may also enable the patient to view the to-do list. An appointment display 728 illustrates upcoming appointments for the patient and may include a widget that enables the patient to schedule future appointments. Upon selecting the to-do list button, screen display 730 lists the various items on the to-do list for the patient. For example, the to-do list may include “Attend Annual Doctor Visit,” “Get your Annual Flu Shot,” “Take Tobacco Cessation Education,” or “Colorectal Cancer Screening.” Each of these items may include widgets that enable the patient to complete the goal or activity on the to-do list.

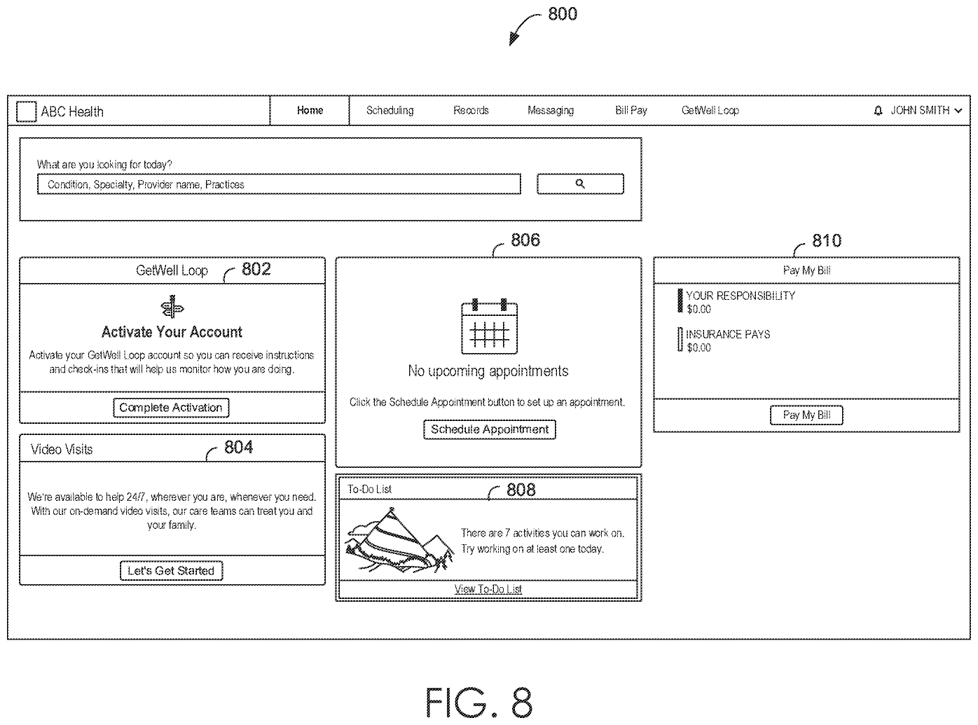

Referring now to FIG. 8, screen display 800 depicts a dashboard for the patient. The dashboard provides various displays and widgets that enable the patient to perform various actions. For example, an activation display 802 allows the patient to activate the longitudinal care plan features for monitoring patient activities and goals. A video visits display 804 allows the patient to initiate on-demand video visits. An appointment display 806 illustrates upcoming appointments for the patient and may include a widget that enables the patient to schedule future appointments. A to-do list display 808 provides an overview of activities the patient can work on and may include a button that allows the patient to view the detailed to-do list. A bill display 810 illustrates an overview of patient responsibility and insurance responsibility for outstanding bills and may include a widget that enables the patient to pay the portion of the bill corresponding to patient responsibility.

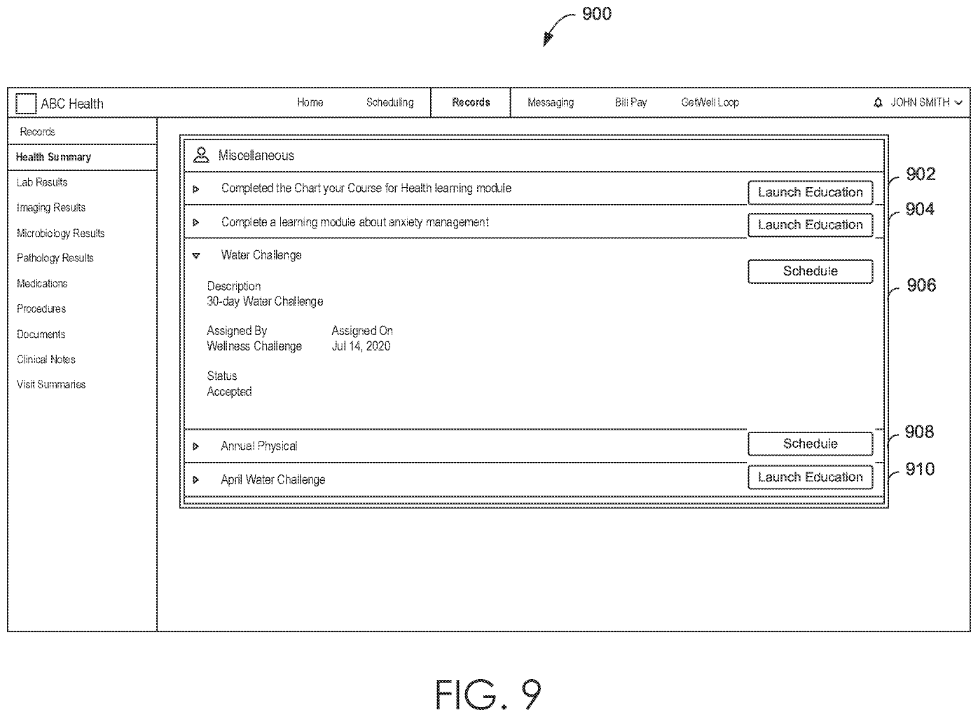

As shown in FIG. 9, screen display 900 depicts the to-do list for the patient. For example, the to-do list includes three learning modules 902, 904, 910 that include widgets enabling the patient to launch the learning modules 902, 904, 910. The to-do list may also include activities 906, 908 such as a water challenge or an annual physical. The activity 906, 908 may include widgets that enable the patient to schedule the activities.

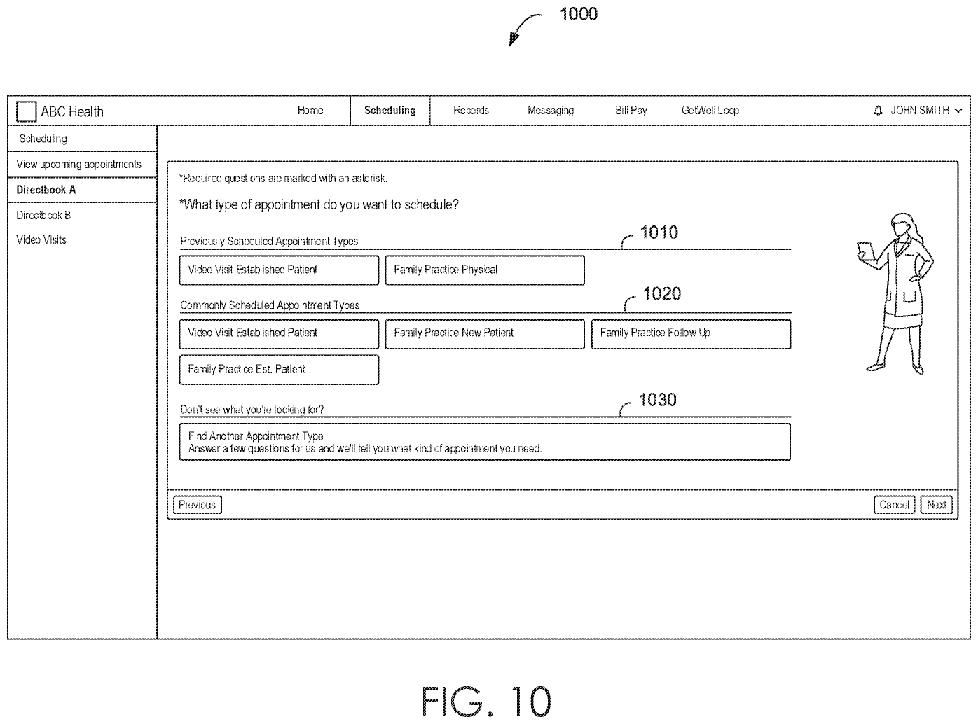

In FIG. 10, screen display 1000 depicts the scheduling integration for the patient. For example, the patient may have utilized the widget that enables the patient to schedule an appointment (as shown in FIG. 7 or 8). Screen display 1000 allows the patient to schedule previously scheduled appointment types 1010, commonly scheduled appointment types 1020, or find other appointment types 1030.

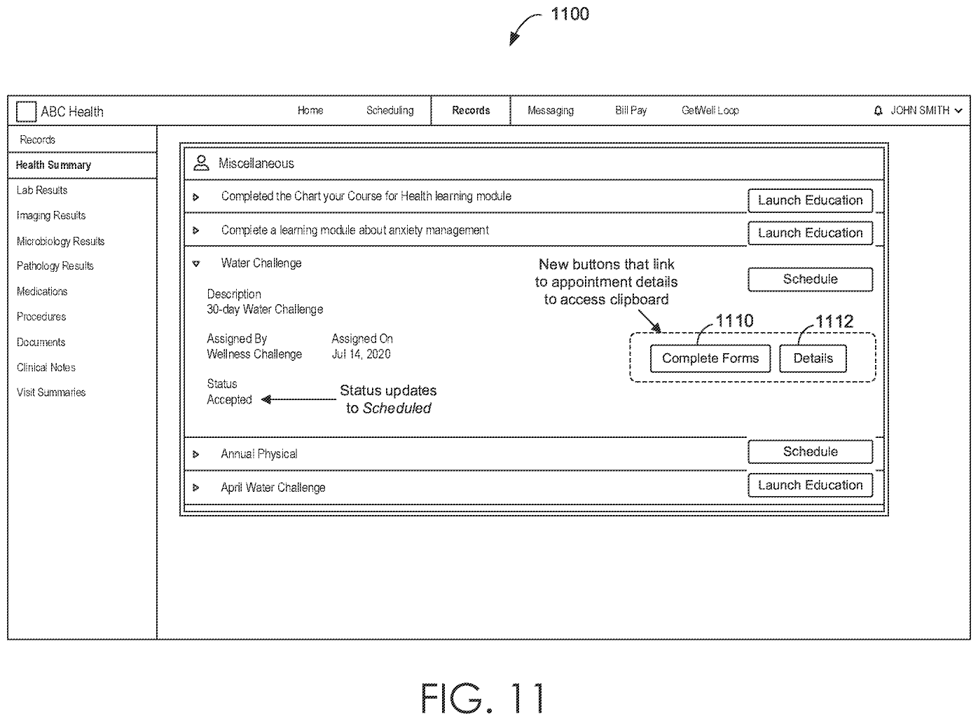

As shown in FIG. 11, screen display 1100 depicts the learning module integration for the patient. For example, the patient may have utilized the widget to launch a particular activity (e.g., the water challenge). Longitudinal care plan system recognizes that the particular activity includes forms that need to be completed by the patient prior to the patient completing the activity. Accordingly, screen display 1100 includes buttons that enable the patient to access the clipboard and complete forms 1110 or view details 1112 corresponding to the activity and/or forms.

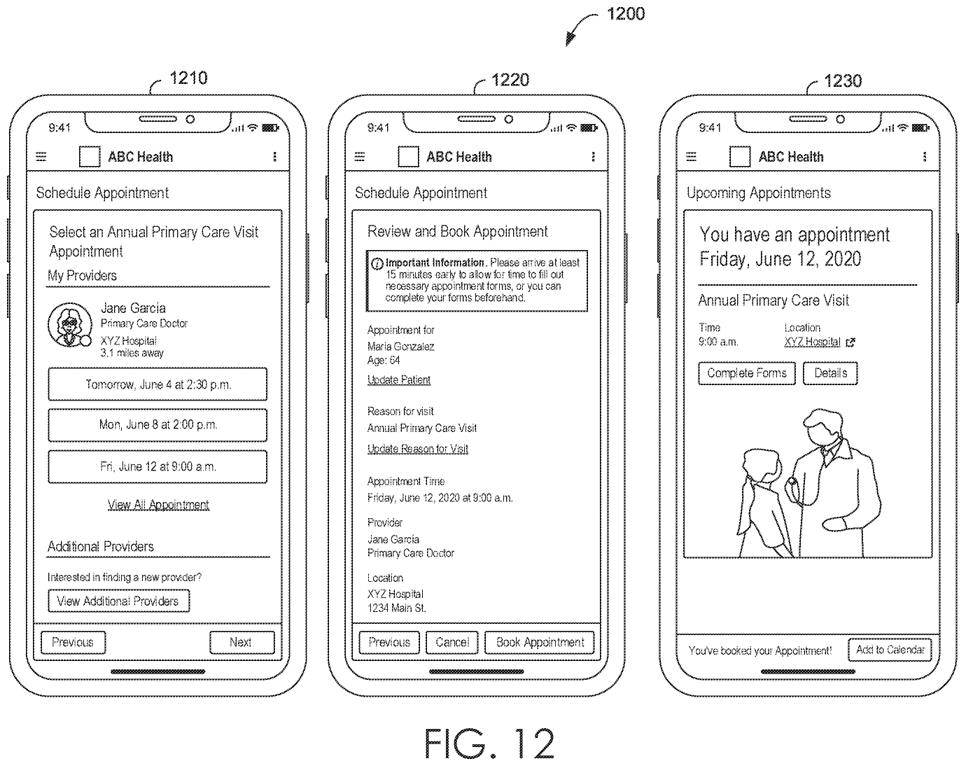

Turning now to FIG. 12, screen displays 1210-1230 depict a progression of user interfaces of a longitudinal care plan system. For example, screen display 1210 illustrates a scheduling interface that enables a patient to select an appointment time with a particular provider or select a different provider. Screen display 1220 illustrates a review and book interface that enables the patient to review details corresponding to the appointment and, upon being satisfied with the details, book the appointment. Screen display 1230 illustrates an upcoming appointments interface that provides buttons to complete forms or view details of any upcoming appointments.

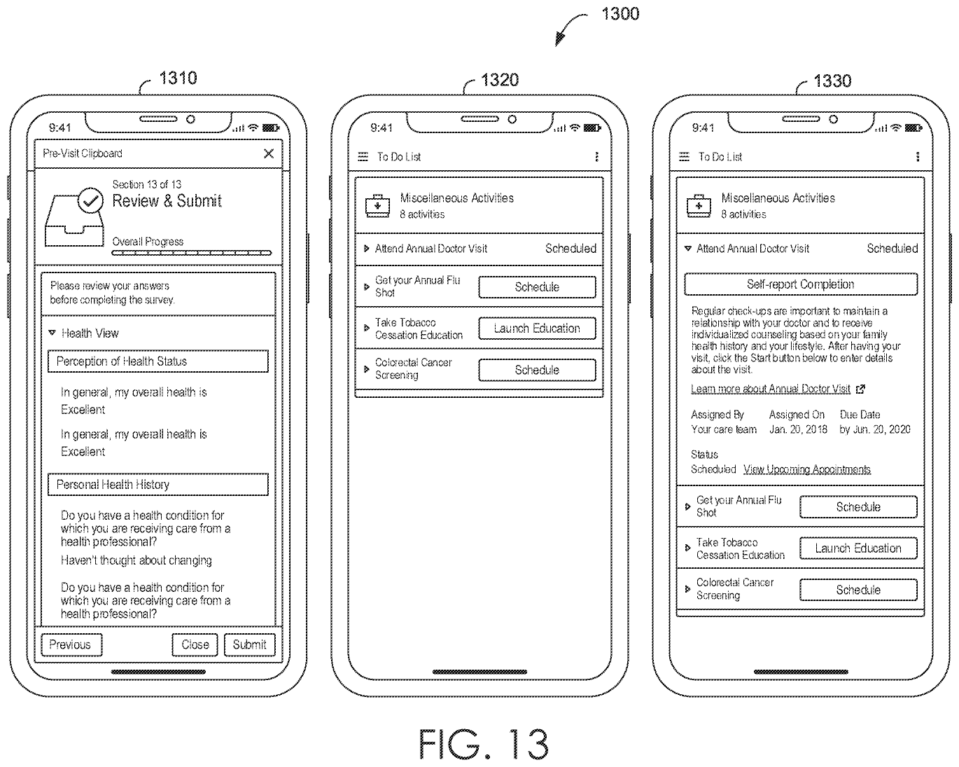

In FIG. 13, screen displays 1310-1330 depict a progression of user interfaces of a longitudinal care plan system. For example, screen display 1310 illustrates a survey interface that enables a patient to complete a survey and/or personal health history. Screen display 1320 illustrates an activities interface that enables the patient to view scheduled activities and/or to-do list items. Screen display 1230 illustrates a details interface corresponding to an activity selected in screen display 1320. As shown, screen display 1230 also enables the patient to select a button to self-report completion of the selected activity which allows longitudinal care plan system to update the to-do list for the patient.

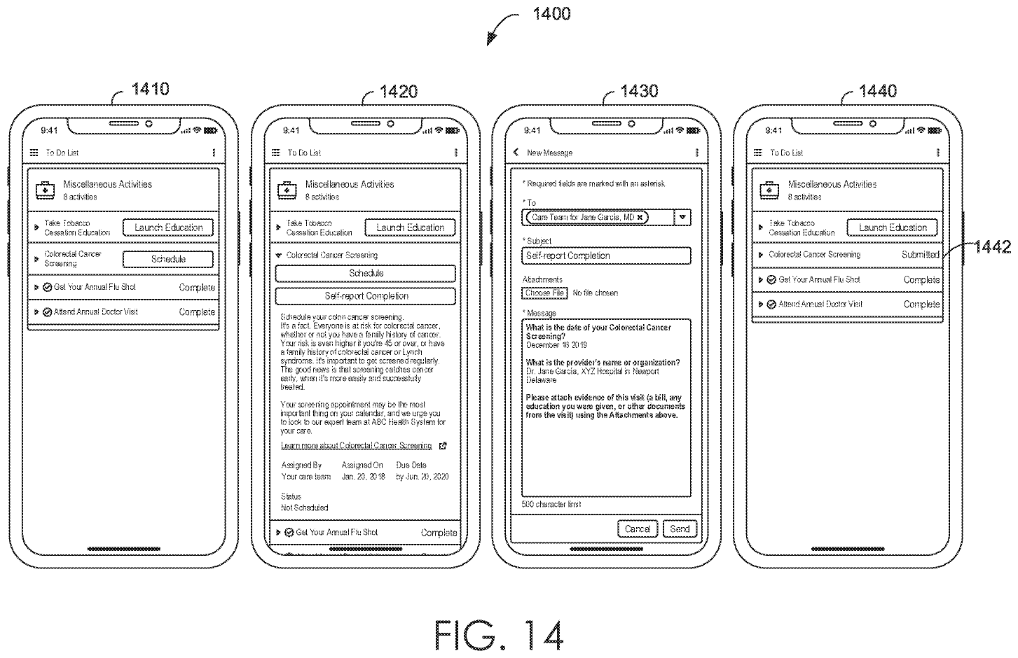

As shown in FIG. 14, screen displays 1410-1440 depict a progression of user interfaces of a longitudinal care plan system. For example, screen display 1410 illustrates an activities interface that enables the patient to view scheduled activities and/or to-do list items. Screen display 1420 illustrates a details interface corresponding to an activity selected in screen display 1410. Upon selecting the button to self-report completion of the selected activity, screen display 1430 illustrates a self-reporting interface. As shown, the self-reporting interface enables the patient to report completion to selected providers, attach relevant files, or include a message corresponding to the selected activity. Upon the patient self-reporting completion of the selected activity, screen display 1440 illustrates the activities interface indicating that the patient has submitted self-reported completion of the activity 1442.

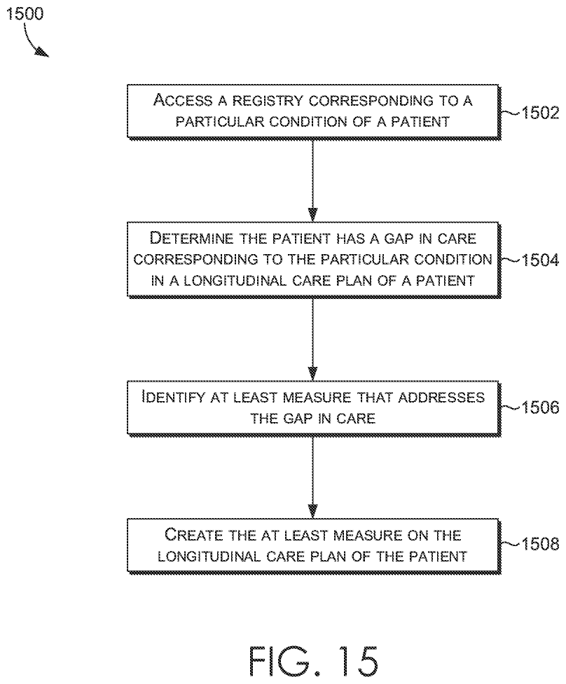

Referring now to FIG. 15, a flow diagram is provided illustrating a method 1700 of creating a longitudinal care plan, in accordance with an embodiment of the present invention, in accordance with embodiments of the present invention. Method 1500 may be performed by any computing device (such as computing device described with respect to FIG. 1) with access to a longitudinal care plan system (such as the one described herein with respect to FIGS. 2-14) or by one or more components of the longitudinal care plan system.

Initially, at step 1502, a registry corresponding to a particular condition of a patient is accessed. At step 1504, it is determined the patient has a gap in care corresponding to the particular condition in a longitudinal care plan of a patient. At least one measure is identified, at step 1506, that addresses the gap in care. At step 1506, the at least one measure is created on the longitudinal care plan of the patient.

In some embodiments, a measure state for the at least one measure is monitored for the patient. If it is determined the patient has completed the measure that addresses the gap in care, the longitudinal care plan of the patient is updated. For example a plurality of sources (e.g., various EMRs, claims systems, pharmacy systems, and the like) may be accessed to determine if the patient has completed the at least one activity.

In some embodiments, an interface is provided via a patient device that enables the patient to self-report the at least one activity as completed. In some embodiments, an interface is provide via a patient device that enables the patient to perform an action to complete the at least one measure. For example, the measure may comprise scheduling an appointment, launching an education module, taking an assessment, or completing a goal. Updates to the longitudinal care plan of the patient may also be provided to a clinician via a clinician device.

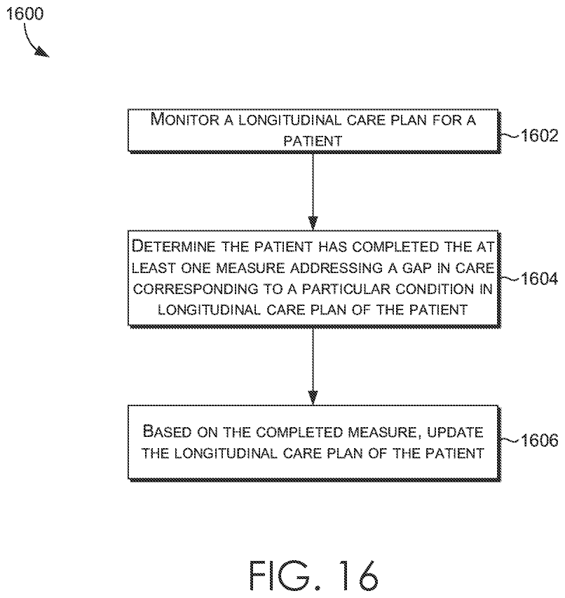

Turning now to FIG. 16, a flow diagram is provided illustrating a method 1600 of updating a longitudinal care plan, in accordance with an embodiment of the present invention, in accordance with embodiments of the present invention. Method 1600 may be performed by any computing device (such as computing device described with respect to FIG. 1) with access to a longitudinal care plan system (such as the one described herein with respect to FIGS. 2-14) or by one or more components of the longitudinal care plan system.

Initially, at step 1602, a measure state corresponding to at least one measure is monitored for a patient. At step 1604, it is determined the patient has completed the at least one measure addressing a gap in care corresponding to a particular condition in the longitudinal care plan of the patient. Based on the measure status, the longitudinal care plan of the patient is updated, at step 1606.

In some embodiments, a registry corresponding to a particular condition of a patient is accessed. If it is determined the patient has a gap in care corresponding to the particular condition in the longitudinal care plan of a patient, at least one measure that addresses the gap in care is identified. Additionally, the at least measure is created on the longitudinal care plan of the patient.

In some embodiments, a plurality of sources is accessed to determine if the patient has completed the at least measure. In other embodiments, an interface is provided via a patient device that enables the patient to self-report the at least measure as completed. An interface may also be provided via the patient device that enables the patient to perform an action to complete the at least measure. The action may comprise scheduling an appointment, launching an education module, taking an assessment, or completing a goal. Updates to the longitudinal care plan of the patient may also be provided to a clinician via a clinician device.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described. Accordingly, the scope of the invention is intended to be limited only by the following claims.

What is claimed is:

1. One or more computer storage media having computer-executable instructions embodied thereon that, when executed by a computer, causes the computer to perform operations, the operations comprising:
accessing a registry corresponding to a particular condition of a patient;
automatically determining the patient has a gap in care corresponding to the particular condition in a longitudinal care plan of a patient;
automatically identifying at least one measure that addresses the gap in care; and
automatically creating the at least one measure on the longitudinal care plan of the patient.

2. The media of claim 1, further comprising monitoring a status of the at least one measure for the patient.

3. The media of claim 2, further comprising determining the patient has completed the at least one measure that addresses the gap in care.

4. The media of claim 3, further comprising, based on the at least one measure being completed, updating the longitudinal care plan of the patient.

5. The media of claim 1, further comprising accessing a plurality of sources to determine if the patient has completed the at least one measure.

6. The media of claim 1, further comprising providing an interface via a patient device that enables the patient to self-report the at least one measure as completed.

7. The media of claim 1, further comprising providing an interface via a patient device that enables the patient to perform an action to complete the at least one measure.

8. The media of claim 7, wherein the action comprises scheduling an appointment, launching an education module, taking an assessment, or completing a goal.

9. The media of claim 1, further comprising providing updates to a care plan of the patient for a clinician.

10. A computerized method comprising:
monitoring a gap in care for a patient;
automatically determining the patient has completed at least one measure addressing a gap in care corresponding to a particular condition in a longitudinal care plan of the patient; and
based on the completed at least one measure, automatically updating the longitudinal care plan of the patient.

11. The method of claim 10, further comprising accessing a registry corresponding to the particular condition of a patient.

12. The method of claim 11, further comprising determining the patient has the gap in care corresponding to the particular condition in the longitudinal care plan of a patient.

13. The method of claim 12, further comprising identifying the at least one measure that addresses the gap in care.

14. The method of claim 13, further comprising creating the at least one measure on the longitudinal care plan of the patient.

15. The method of claim 10, further comprising accessing a plurality of sources to determine if the patient has completed the at least one measure.

16. The method of claim 10, further comprising providing an interface via a patient device that enables the patient to self-report the at least one measure as completed.

17. The method of claim 10, further comprising providing an interface via a patient device that enables the patient to perform an action to complete the at least one measure.

18. The method of claim 17, wherein the action comprises scheduling an appointment, launching an education module, taking an assessment, or completing a goal.

19. The method of claim 10, further comprising providing updates to a care plan of the patient for a clinician.

20. A system comprising:
a processor; and
a computer storage medium storing computer-usable instructions that, when used by the processor, cause the processor to:
access a registry corresponding to a particular condition of a patient;
automatically determine the patient has a gap in care corresponding to the particular condition in a longitudinal care plan of a patient;
automatically create at least one measure on the longitudinal care plan of the patient to address the gap in care;
monitor a status of the at least one measure for the patient;
determine the patient has completed the at least one measure that addresses the gap in care; and
based on the completed at least one activity, automatically update the longitudinal care plan of the patient.
